(19) United States
(12) Patent Application Publication
(10) Pub. No.: US 2005/0035301 A1
(43) Pub. Date: Feb. 17, 2005

(54) Apparatus and a method for improving sanitation effectiveness of UV light

(76) Inventor: Xiaoling Wang, Princeton Junction, NJ (US)

Correspondence Address: Mr. Walter J. Tencza Jr., Suite 3, 10 Station Place, Metuchen, NJ, 08840, US

(21) Appl. No.: 10/681,454

(22) Filed: Oct. 8, 2003

Related U.S. Application Data
(60) Provisional application No. 60/493,988, filed on Aug. 11, 2003.

(51) Int. Cl. A61L 2/10; A61L 9/20; C02F 1/32

(52) U.S. Cl. 250/436; 250/437

(57) ABSTRACT

An apparatus and a method is disclosed comprising a pipe based air or water chamber that can significantly increase the travel time of a substance such as air or water flowing through the air or water chamber. The pipes are constructed using UV transparent materials so that UV light can radiate the air or water inside of the pipes effectively. The pipes can have various shapes and forms as long as they are connected to each other.

### CROSS REFERENCE TO RELATED APPLICATIONS

The present application claims the priority of a provisional application filed on Aug. 7, 2003, titled AN APPARATUS AND A METHOD FOR IMPROVING SANITATION EFFECTIVENESS OF UV LIGHT inventor and applicant Xiaoling Wang, serial No. currently unknown.

### FIELD OF THE INVENTION

This invention relates to the field of systems and methods for air or water sterilization and sanitation using ultraviolet (“UV”) light.

### BACKGROUND OF THE INVENTION

Recently, due to the increased risks of bio-terrorism and new diseases, such as the Severe Acute Respiratory Symptom (SARS), efficient air and water sterilization systems using ultraviolet (UV) light are very helpful. However, most existing air and water sterilization systems using UV light are not very efficient. They have in general a simple air or water chamber with one or more UV sources. The air or water flows into the chamber from one end, travels quickly through the chamber, and exits at the other end of the chamber. The travel time of the air or water through the chamber determines in general the radiation time of the air or water by the UV light. The longer the air travel time, the longer the UV radiation time, and hence the better the effectiveness of killing germs using the UV light. It is desirable to significantly increase the air or water travel time through a given size of air or water chamber for significantly improving effectiveness of killing germs using UV light.

### SUMMARY OF THE INVENTION

The present invention, in one or more embodiments, introduces a pipe based air or water chamber that can significantly increase the travel time of air or water through the air or water chamber. The pipes are constructed using UV transparent materials so that UV light can radiate the air or water inside of the pipes effectively. The pipes can have various shapes and forms as long as they are connected to each other. The pipes are connected with air-tight or water-tight connection devices so that the air and water can flow freely through the connected pipes.

### BRIEF DESCRIPTION OF THE DRAWINGS

FIG. 1 is a perspective view schematically illustrating the overall structure of one embodiment of the present invention for a sterilization chamber using UV light;

FIG. 2A is a perspective view schematically illustrating a chamber enclosure with inflow and outflow openings located on opposite sides;

FIG. 2B is a perspective view schematically illustrating a chamber enclosure with a rectangular cross section and with inflow and outflow openings located on opposite sides;

FIG. 2C is a perspective view schematically illustrating a chamber enclosure with two inflow and two outflow openings located on opposite sides;

FIG. 2D is a perspective view schematically illustrating a chamber enclosure with multiple inflow and outflow openings located on different sides and in different locations;

FIG. 3A is a perspective view schematically illustrating a circular cross section for a chamber enclosure with six pipes and one UV light source;

FIG. 3B is a perspective view schematically illustrating a rectangular cross section for a chamber enclosure with six pipes and two UV light sources;

FIG. 3C is a perspective view schematically illustrating a rectangular cross section for a chamber enclosure with eight pipes and six UV light sources;

FIG. 3D is a perspective view schematically illustrating a circular cross section for a chamber enclosure with fifteen pipes and five UV light sources;

FIG. 4A is a perspective view schematically illustrating a U-shaped pipe;

FIG. 4B is a perspective view schematically illustrating a W-shaped pipe;

FIG. 4C is a perspective view schematically illustrating a pipe with more turns than in FIG. 4B;

FIG. 4D is a perspective view schematically illustrating a spiral shaped pipe with multiple turns;

FIG. 4E is a perspective view schematically illustrating a rectangular pipe;

FIG. 4F is a cross sectional view illustrating the rectangular pipe shown in FIG. 4E with multiple internal rectangular isolation plates;

and FIG. 5 is a sectional view schematically illustrating a pipe device and a cover device for connecting to one end of each of a plurality of pipes of the pipe device.

### DETAILED DESCRIPTION OF THE INVENTION

The present invention, in one or more embodiments, introduces a pipe device that is transparent to ultraviolet (UV) light for significantly increasing the travel time of air or water through an air or water chamber.

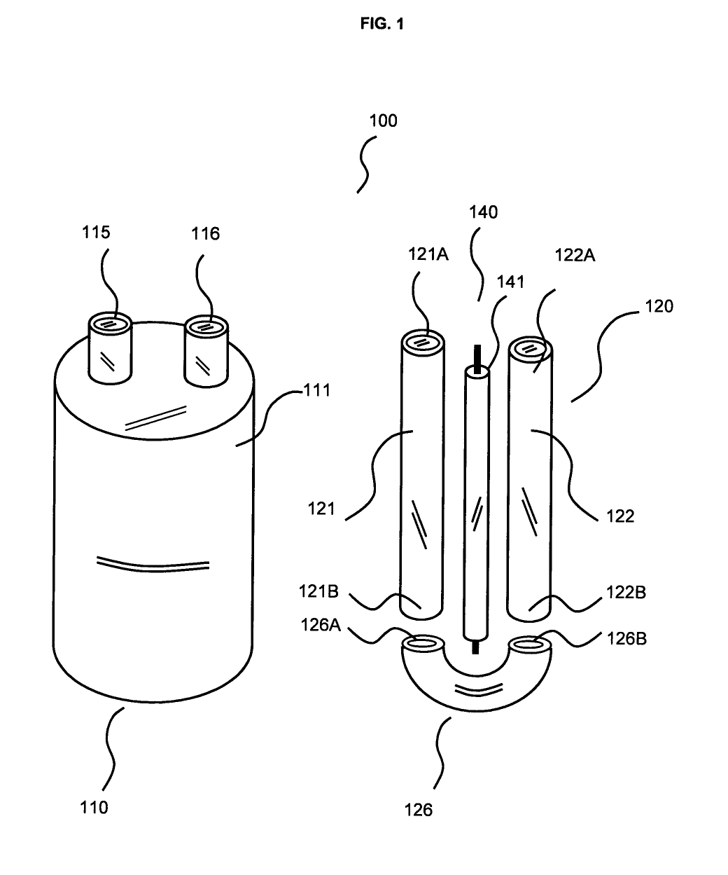

A perspective view of a system, apparatus, and method according to one embodiment of the present invention is shown in FIG. 1. FIG. 1 shows an apparatus 100 comprised of an air or water chamber enclosure 110, a pipe device 120, and a UV lighting device 140. Since the same system, apparatus, and method will work for both air and water sterilization, in the following descriptions, we will only mention air instead of both air and water for clarity. The chamber enclosure 110 is typically comprised of a housing 111, an inflow opening 115, and an outflow opening 116. The housing 111 is commonly made using plastic and/or metal materials. The pipe device 120 is typically comprised of a plurality of pipes, such as pipes 121 and 122, and pipe connectors, such as connector 126, which may connect the pipes 121 and 122 together. As shown in FIG. 1, two ends 126A and 126B of the connector 126 may be connected to ends 121B and 122B of the two pipes, respectively. The pipe device 120 may be connected to the inflow opening 115 and the outflow opening 116 by connecting ends 121A and 122A to the bottom ends of inflow opening 115 and outflow opening 116, respectively. One of the most important properties of the pipes 121 and 122, is that the pipes 121 and 122 be transparent to UV light. This allows UV light to penetrate the pipes 121 and 122 and radiate the air or water inside of the pipes 121 and 122 effectively. The pipe connectors 126 can be either UV light transparent or not. The UV lighting device 140 is typically comprised of one or more UV light sources, such as a UV light source 141 shown in FIG. 1. In addition, the apparatus 100 may further contain a UV light reflecting device (not shown in FIG. 1 for clarity) that surrounds the pipe device 120 and the UV lighting device 140 for increasing the effectiveness of the UV radiation of the air or water inside of the pipes 121 and 122. It is also possible to make the inner surface of the chamber enclosure 110, UV reflective so that the UV light reflecting device can be omitted.

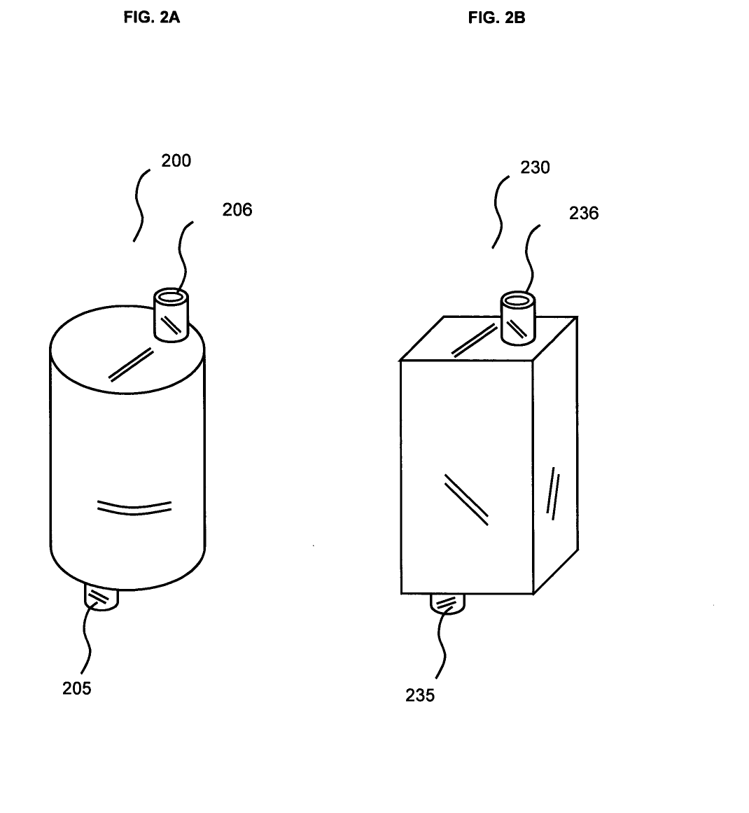

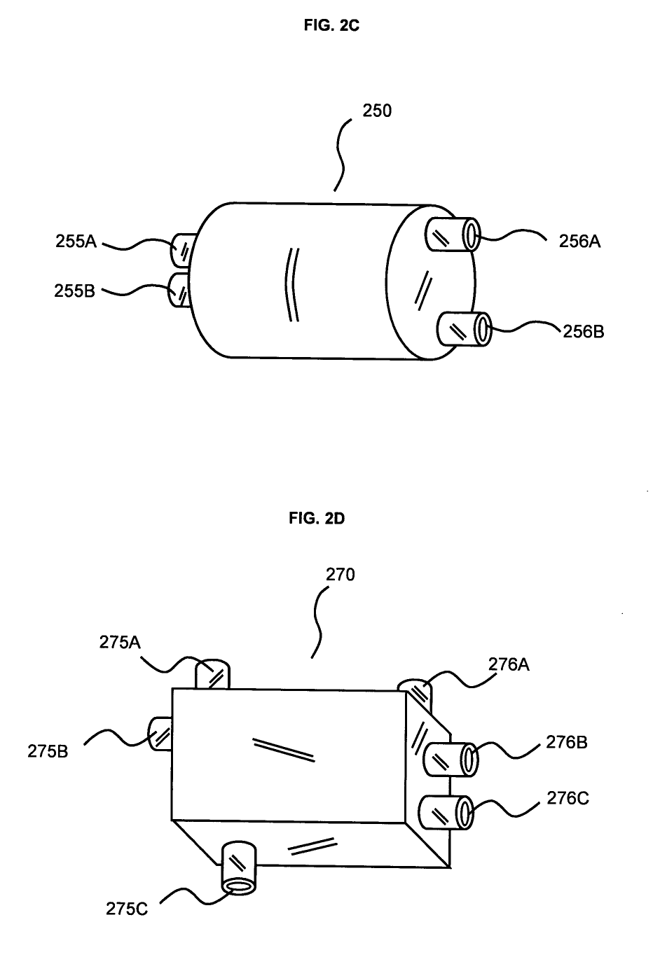

Instead of the chamber enclosure 110 shown in FIG. 1, a chamber enclosure may be provided in various shapes and forms. Four examples are shown in FIGS. 2A, 2B, 2C and 2D. As shown in FIG. 2A, a chamber enclosure 200 has one inflow opening 205 and one outflow opening 206. In this case, the openings 205 and 206 are not located on the same side of the chamber enclosure 200, as is the case for openings 115 and 116 of chamber 110 shown in FIG. 1. FIG. 2B depicts another variation of shape for a chamber enclosure. Instead of a circular or elliptical sectional shape for a chamber enclosure, a chamber enclosure 230 is provided which has a rectangular shape with an inflow opening 235 and an outflow opening 236 located on opposite sides of the chamber enclosure 230. FIG. 2C shows a chamber enclosure 250 with two inflow openings 255A and 255B as well as two outflow openings 256A and 256B, located on opposite sides of the chamber enclosure 250. Multiple inflow openings and outflow openings may be used if more than one air or water flows need to be radiated with UV light. FIG. 2D shows a chamber enclosure 270 with three inflow openings 275A, 275B, and 275C as well as three outflow openings 276A, 276B and 276C, located at various locations of the chamber enclosure 270. By accepting multiple inflows and outflows, the same chamber with one UV lighting device may be used for sanitizing multiple water or air flows simultaneously.

In addition to elliptical (including circular as a special case) and rectangular (including square as a special case) sectional shapes for a chamber enclosure, there are some other common sectional shapes, such as a ring shape or a polygonal shape, that also may be useful. Special shapes and forms for a chamber enclosure may be useful for particular applications or to be embedded into other machines that have a special shape constraint. Furthermore, multiple inflow and outflow openings may be used to radiate multiple flows simultaneously. Such changes and modifications may reasonably be included within the scope of the present invention.

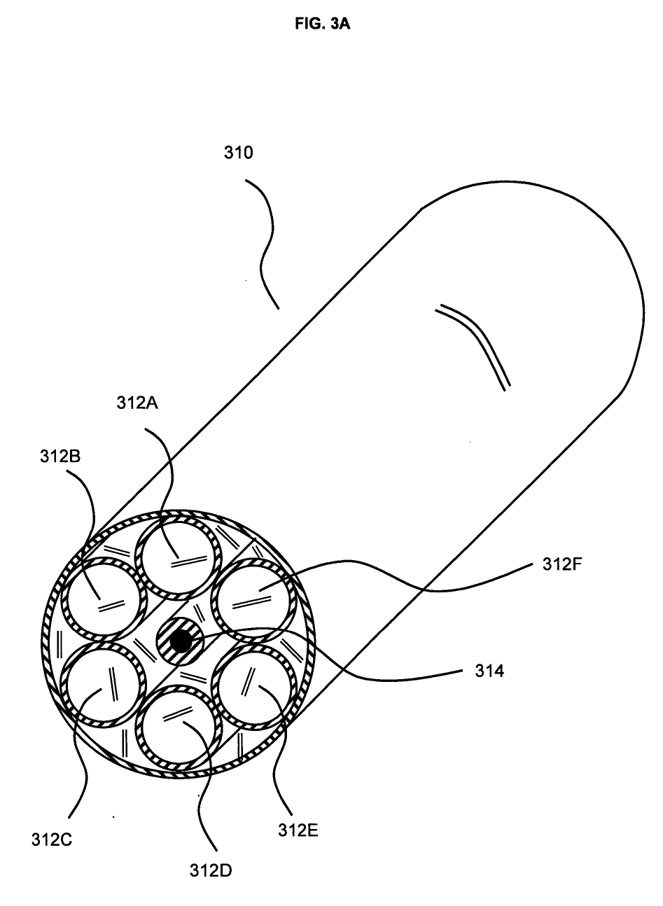

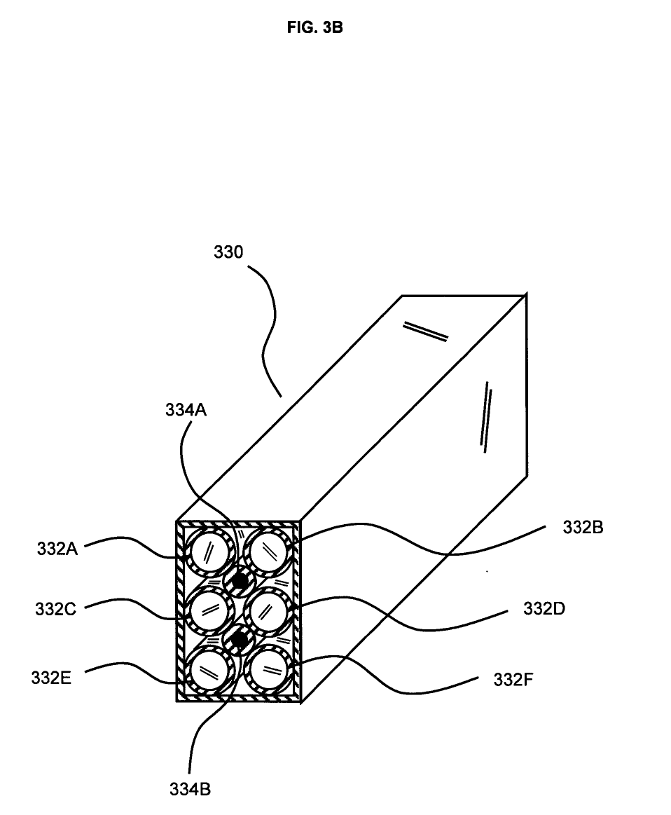

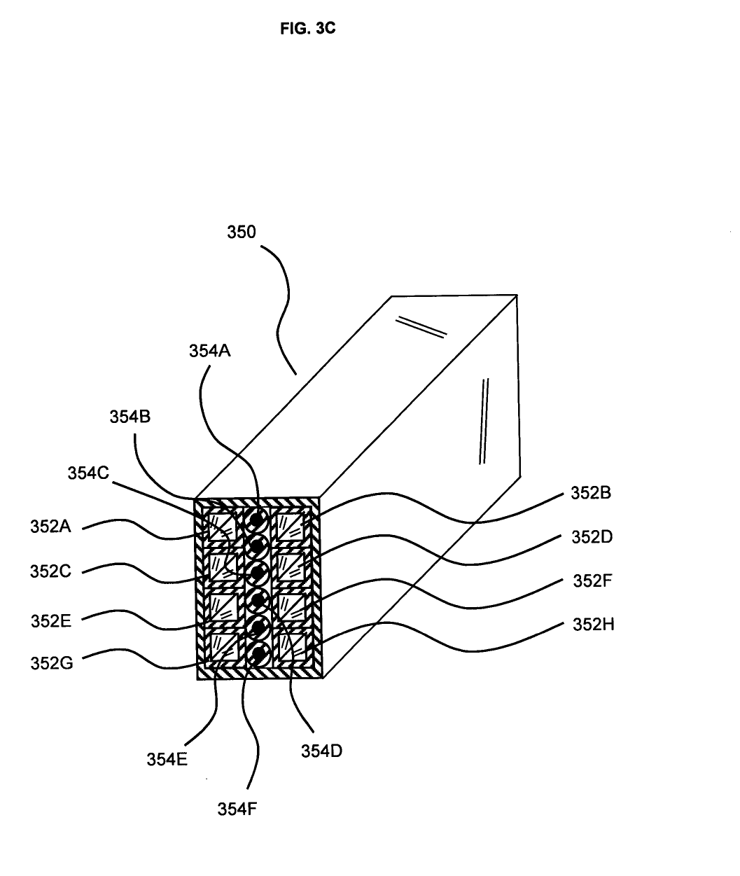

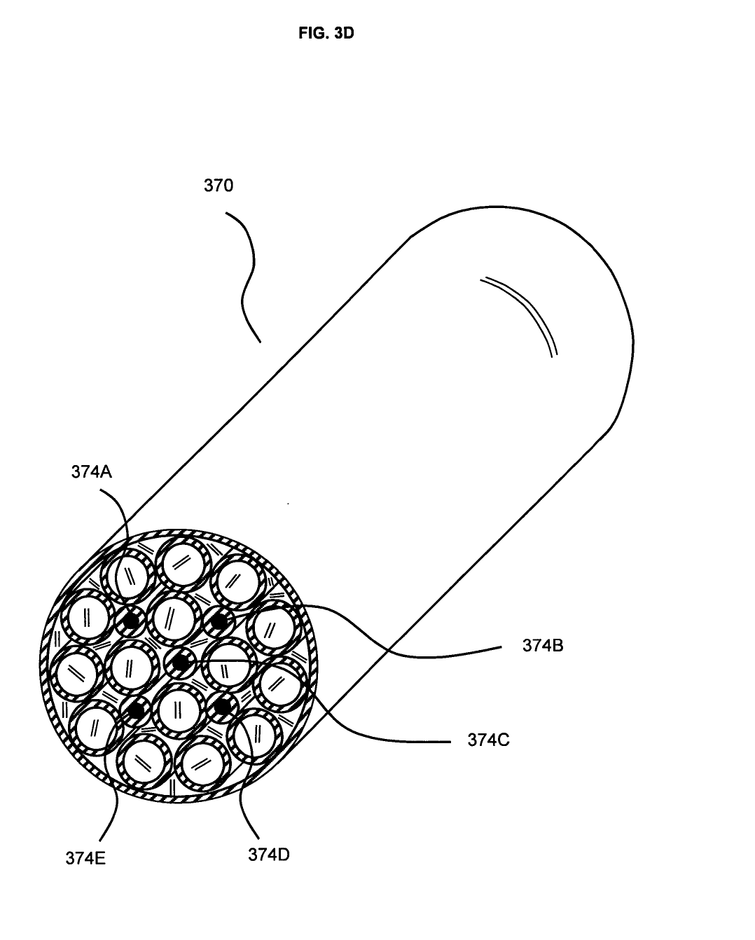

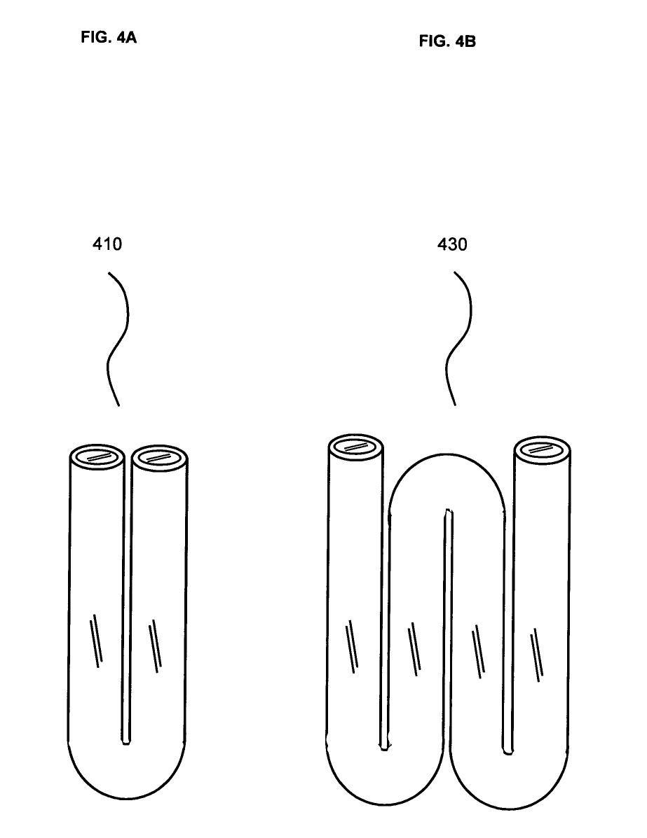

The pipe device 120 shown in FIG. 1 can take various shapes and forms, and may be rigid or flexible. Depending on the geometry of the chamber enclosure 110 as well as the shape and position of the UV lighting device 140, the pipe device 120 can be designed specifically to allow efficient UV radiation and long travel time. Four examples of the pipe arrangement with one or more UV light sources are shown in FIGS. 3A-D. FIG. 3A shows an arrangement comprised of a chamber 310 containing six pipes 312A-F and one UV light source 314. The total travel time of air or water flowing inside the pipes 312A-F are therefore approximately six times longer than the time needed for the air or water to simply travel through the chamber 310 without the pipes 312A-F. The total UV radiation with the arrangement of FIG. 3A is therefore roughly six times the total UV radiation which would occur without the pipes 312A-F. FIG. 3B depicts another arrangement with six pipes 332A-F and two UV light sources 334A-B placed into a chamber enclosure 330 with a rectangular cross section. The total travel time of air or water flowing inside the pipes 332A-F is therefore approximately six times longer than the time needed for simply traveling through the chamber without the pipes 332A-F. With the two UV light sources 334A and 334B, the total radiation applied to the air or water inside of the pipes 332A-F is therefore twelve times more than a chamber without a pipe and with only one UV light source. The radiation amount may further be increased if more pipes and more UV light sources are used, such as the arrangement shown in FIG. 3C. In this case, a chamber 350 with eight pipes 352A-H with rectangular cross section and 6 UV light sources 354A-F, may deliver forty-eight times more UV radiation to the air or water flowing through the pipes 352A-H than a chamber without the pipes and with only one UV light source. FIG. 3D shows another arrangement with fifteen pipes and five UV light sources 374A-E, the chamber 370 in this case may deliver seventy-five times more UV radiation to the air or water flowing through the chamber 370 than a chamber without the pipes and with only one UV light source.

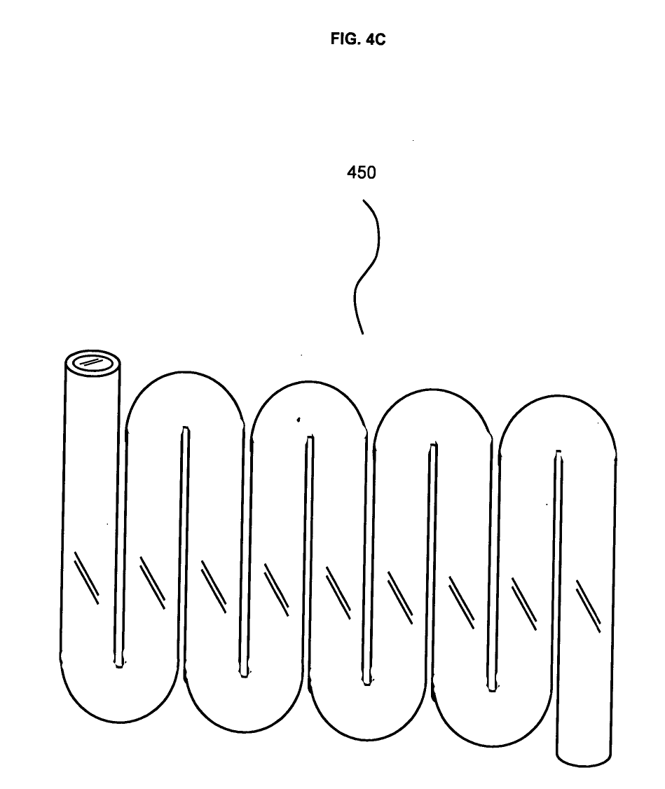

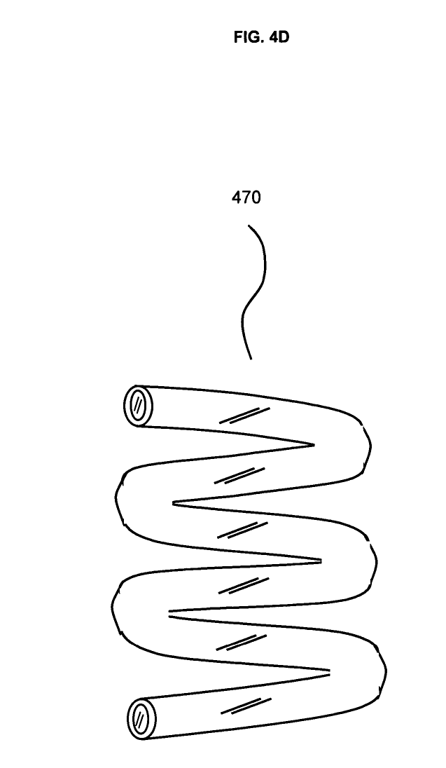

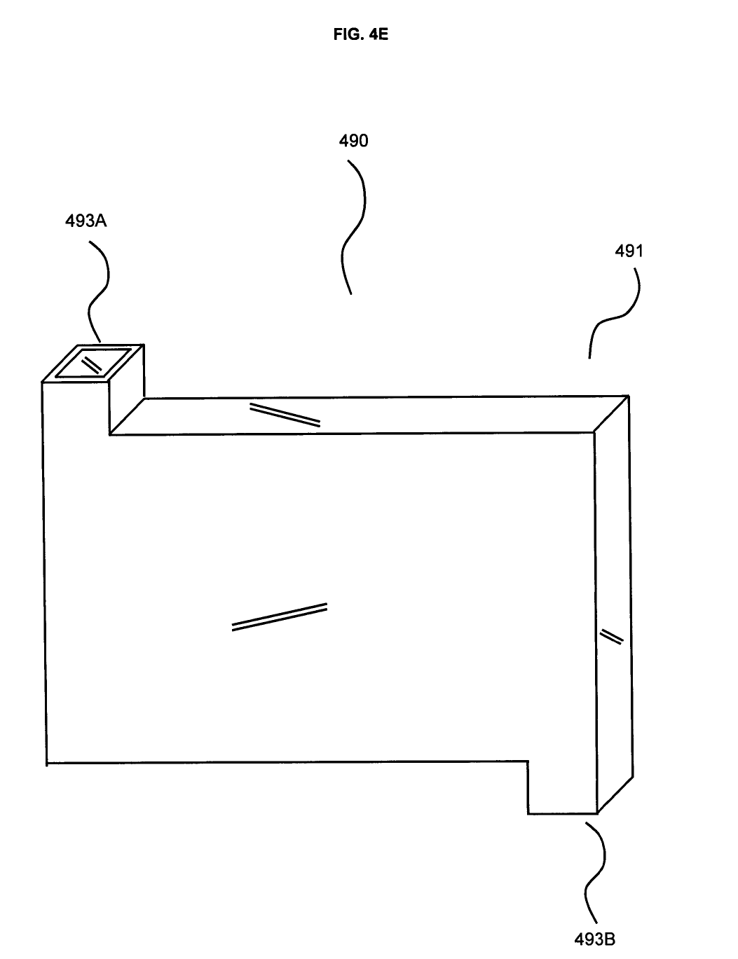

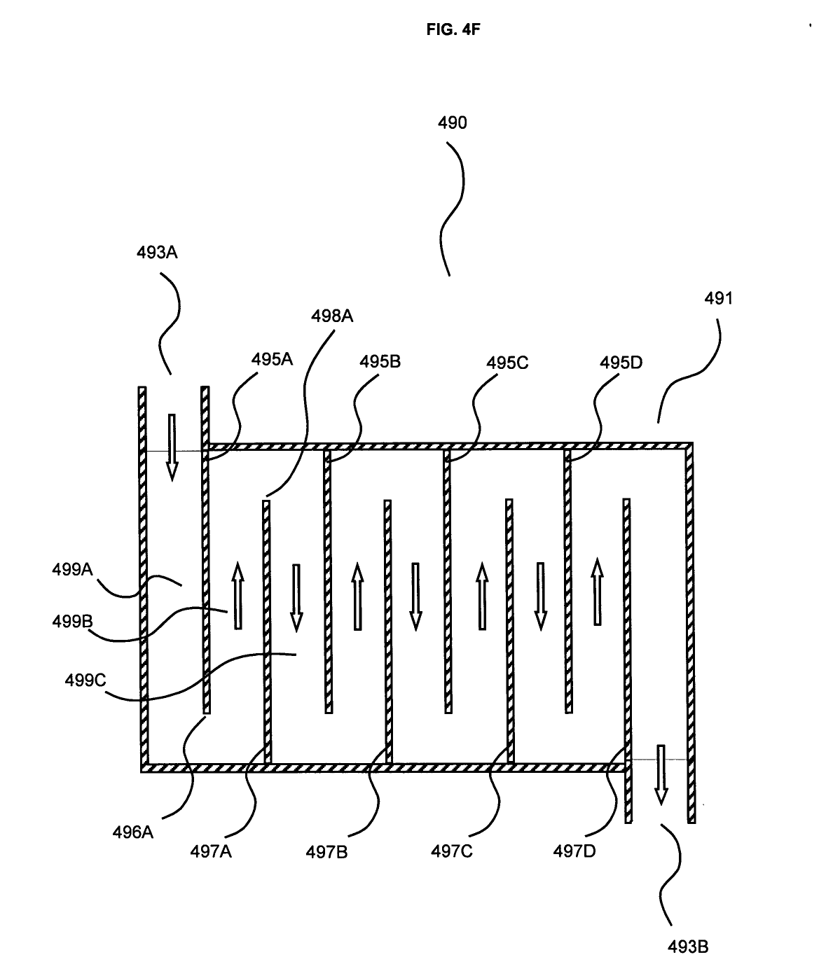

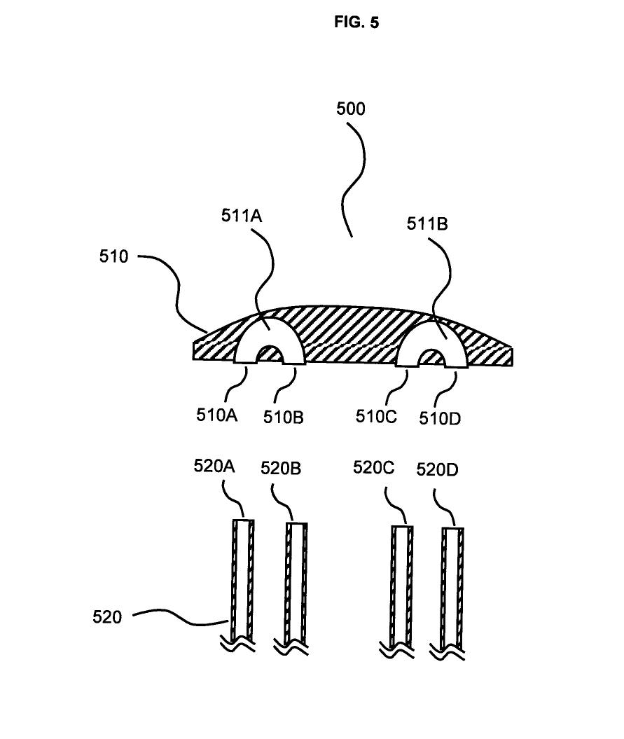

Instead of the pipe device 120 shown in FIG. 1, a pipe device 410 shown in FIG. 4A may be provided, which may be comprised of just one U-shaped pipe without the connector 126. FIG. 4B depicts a W-shaped pipe 430 without a connector, which may be used instead of pipe device 120. If longer radiation time is desired, a pipe device with more turns than the U-shaped pipe 410 or W-shaped pipe 430 may be employed, such as the pipe device 450 shown in FIG. 4C. A spiral shaped pipe 470 shown in FIG. 4D with multiple turns may also be used.

A rectangular-shaped pipe device 490 with multiple internal rectangular isolation plates (to be shown in FIG. 4F later) is shown in FIG. 4E. It has a rectangular pipe 491 with an inflow opening 493A and the outflow opening 493B.

The internal structure of the rectangular-shaped pipe device 490 may be seen more clearly in the cross sectional view shown in FIG. 4F. The rectangular isolation plates 495A-D are placed inside of a large rectangular pipe 491 in such a way that air or water may only flow from the bottom part of plates from one region to the neighboring one. For example, air can flow from region 499A to region 499B under the bottom edge 496A of the isolation plate 495A. In contrast, the rectangular isolation plates 497A-D enable the air or water flow from their top part. For example, air can flow from region 499B to region 499C over the top edge 498A of the plate 497A.

The inflow opening 493A and the outflow opening 493B allow air or water flow to enter and exit the rectangular-shaped pipe device 490. The arrows indicate the flow directions within the rectangular-shaped pipe device 490.

When a plurality of pipes is used in a pipe device, a plurality of connectors may be used to connect the plurality of pipes. If the pipes to be connected are similar in length, multiple connectors may be molded together to form a connecting cover that may cover and connect all pipes at one end of each of the pipes. FIG. 5 shows a sectional view of a connecting cover 510 with four openings 510A-D and a pipe device 520 with four pipe openings 520A-D on one end to be connected. The two openings 510A and 510B are actually connected through a built-in connector 511A located inside and part of the cover 510. Similarly, the two openings 510C and 510D are also connected by a built-in connector 511B inside the cover 510. The built-in connectors 511A and 511B are actually molded together with the connecting cover 510 so that they become one piece. When the connecting cover 510 is put together with the pipe device 520, openings 520A, 520B, 520C, and 520D on one end of the pipe device 520 are connected to corresponding openings 510A, 510B, 510C, and 510D in the connecting cover 510, respectively. By using the connecting cover 510, the pipes 520A and 520B are now connected through integrated connector 511A without using a separate connector. Similarly, the pipes 520C and 520D are now also connected through integrated connector 511B without using a separate connector. When more pipes need to be connected, a connecting cover with more openings and more integrated connectors may be deployed to connect the pipe openings. If there are openings on the other end of the pipes to be connected, one more connecting covers may be used to cover and connect the pipes. The use of a connecting cover with integrated connectors may reduce the manufacturing cost.

The apparatus and method disclosed in the present invention may be used in various air sterilization systems. The apparatus according to the present invention may either be used as a stand-alone air sterilization device or be integrated into large air conditioning, controlling and processing systems. In addition to central air conditioning systems, the apparatus and the method according to the present invention may also be used in various stand-alone air conditioning devices. These devices are widely used for home and room temperature conditioning. Furthermore, the apparatus and the method according to the present invention may also be used and integrated into most indoor air processing devices, such as air dust particle removing devices, smoke removing devices, air odor removing devices, air ionization devices, to form multi-purposed indoor air processing devices. When the apparatus and the method according to the present invention is used in a stand-alone air processing system with no air pumping (sucking) device, an air pumping (sucking) device may be added and connected to the apparatus for pumping or sucking air into the pipe device for receiving UV radiations. The apparatus and the method according to the present invention may also be used for disinfecting drinking water using UV light.

Although the invention has been described by reference to particular illustrative embodiments thereof, many changes and modifications of the invention may become apparent to those skilled in the art without departing from the spirit and scope of the invention. It is therefore intended to include within this patent all such changes and modifications as may reasonably and properly be included within the scope of the present invention's contribution to the art.

1. An apparatus comprising
a pipe device; and
an ultraviolet lighting device;
wherein a substance can flow through the pipe device; and
wherein the ultraviolet lighting device delivers ultraviolet radiation to the substance while it is inside the pipe device.

2. The apparatus of claim 1 wherein
the substance is air.

3. The apparatus of claim 1 wherein
the substance is water.

4. The apparatus of claim 1 further comprising
an enclosure;
wherein the pipe device is placed inside the enclosure;
wherein the ultraviolet lighting device is placed inside the enclosure;
wherein the enclosure is not ultraviolet transparent so that the ultraviolet radiation from the ultraviolet lighting device is contained within the enclosure.

5. The apparatus of claim 4 wherein
the enclosure has one inflow opening through which the substance can enter the enclosure and one outflow opening through which the substance can exit the enclosure.

6. The apparatus of claim 5 wherein
the enclosure has an inside surface which is ultraviolet reflective.

7. The apparatus of claim 4 wherein
the enclosure has a circular cross section.

8. The apparatus of claim 4 wherein
the enclosure has an elliptical cross section.

9. The apparatus of claim 4 wherein
the enclosure has a square cross section.

10. The apparatus of claim 4 wherein
the enclosure has a rectangular cross section.

11. The apparatus of claim 4 wherein
the enclosure has a polygonal cross section.

12. The apparatus of claim 4 wherein
the enclosure is further comprised of a plurality of inflow and outflow openings.

13. The apparatus of claim 12 wherein
the plurality of the inflow and the outflow openings are connected to the pipe device.

14. The apparatus of claim 1 wherein
the pipe device is comprised of one pipe that is ultraviolet transparent.

15. The apparatus of claim 1 wherein
the pipe device is comprised of a plurality of pipes that are ultraviolet transparent.

16. The apparatus of claim 15 wherein
each of the plurality of pipes has a circular cross section.

17. The apparatus of claim 15 wherein
each of the plurality of pipes has an elliptical cross section.

18. The apparatus of claim 15 wherein
each of the plurality of pipes has a square cross section.

19. The apparatus of claim 15 wherein
each of the pipes has a rectangular cross section.

20. The apparatus of claim 15 wherein
each of the pipes has a polygonal cross section.

21. The apparatus of claim 1 wherein
the pipe device is comprised of a plurality of pipes and connectors; and
wherein each of the pipes is connected to at least one other pipe by a connector.

22. The apparatus of claim 1 wherein
the pipe device is comprised of a plurality of pipes and two connecting covers;
wherein the two connecting covers contain a plurality of connectors connecting all pipes at each end.

23. The apparatus of claim 1 wherein
the pipe device is comprised of a U-shaped pipe.

24. The apparatus of claim 1 wherein
the pipe device is comprised of a W-shaped pipe.

25. The apparatus of claim 1 wherein
the pipe device is comprised of a pipe containing multiple turns.

26. The apparatus of claim 1 wherein
the pipe device is comprised of a long and bended pipe in a spiral shape with multiple turns.

27. The apparatus of claim 1 wherein
the pipe device is comprised of a plurality of pipes each having a different shape.

28. An apparatus comprising
an enclosure; and
a pipe device; and
a ultraviolet lighting device;
wherein the enclosure is comprised of a plurality of inflow openings and a plurality of outflow openings;
wherein the pipe device is placed inside of the enclosure and is connected to the plurality of inflow openings and to the plurality of outflow openings;
wherein the ultraviolet lighting device is placed inside of the enclosure; and
wherein the ultraviolet lighting device delivers ultraviolet radiation to the flow inside the pipe device.

29. The apparatus of claim 28 wherein
the inside surface of the enclosure is reflects ultraviolet light.

30. The apparatus of claim 28 wherein
the pipe device is comprised of a plurality of pipes each of which are transparent to ultraviolet light.

31. The apparatus of claim 28 wherein
the pipe device is comprised of a plurality of pipes, each of which has a circular cross section.

32. The apparatus of claim 28 wherein
the pipe device is comprised of a plurality of pipes and connectors;
each of the pipes is connected to at least one other pipe by at least one connector.

33. The apparatus of claim 28 wherein
the pipe device is comprised of a plurality of pipes and first and second connecting covers;
wherein the two connecting covers containing a plurality of connectors connecting all pipes at each end.

34. The apparatus of claim 28 wherein
the pipe device is comprised of a U-shaped pipe.

35. The apparatus of claim 28 wherein
the pipe device is comprised of a W-shaped pipe.

36. The apparatus of claim 28 wherein
the pipe device is comprised of a pipe containing multiple turns.

37. The apparatus of claim 28 wherein
the pipe device is comprised of a long and bended pipe in a spiral shape with multiple turns.

38. The apparatus of claim 28 wherein
the pipe device is comprised of a first pipe having a first shape and a second pipe having a second shape, the first shape differing from the second shape.

39. A method comprising the steps of
using pipes which are transparent to ultraviolet light to force a substance flowing through the pipes to travel longer inside a chamber containing an ultraviolet light source for increasing the time during which the substance is exposed to ultraviolet radiation; wherein
the pipes are connected.
